(19) United States
(12) Patent Application Publication
(10) Pub. No.: US 2010/0016719 A1
(43) Pub. Date: Jan. 21, 2010

(54) ADAPTIVE REGULATION OF ACOUSTIC OUTPUT POWER IN MEDICAL ULTRASOUND IMAGING

(75) Inventors: Paul Freiburger, Seattle, WA (US); Leixiang Fan, Sammamish, WA (US); John Dennis, Renton, WA (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: SIEMENS MEDICAL SOLUTIONS USA, INC., Malvern, PA (US)

(21) Appl. No.: 12/174,514

(22) Filed: Jul. 16, 2008

(51) Int. Cl. A61B 8/00

(52) U.S. Cl. 600/443

(57) ABSTRACT

Acoustic output power is adaptively regulated in medical diagnostic ultrasound imaging. Given transmit settings, the acoustic power and/or another setting may be altered to avoid violating a limit set by regulation. The limit is based, at least in part, on an assumed attenuation. By measuring the attenuation, such as by identifying a type of tissue, the power and/or limit may be altered or adapt. By accounting for the attenuation in the region to be scanned, a greater output power may be possible while still providing the same level of safety.

### BACKGROUND

The present embodiments relate to medical diagnostic ultrasonic imaging, and in particular, to systems that adaptively regulate acoustic output power.

Medical ultrasound imaging systems limit their acoustic output power pursuant to Food and Drug Administration (FDA), Underwriters Laboratories (UL), International Electrotechnical Commission (IEC), and/or other safety regulations. Four example regulatory limits are the mechanical index (MI), thermal index (TI), intensity spatial peak temporal average (Ispta), and transducer temperature. The IEC standards require that an ultrasound transducer temperature not exceed a predetermined limit of 43 degrees C. to avoid burns. For mechanical index, the FDA limits the peak-negative acoustic pressure, such as at or near a focal point, to a (frequency-dependent) value, such as 1.9 MPa (at 1 MHz). For the thermal index, the power over time is limited to avoid substantial heating of tissue.

The acoustic power, frequency, pulse repetition rate, aperture size, and other transmit characteristics are adjusted to avoid exceeding the regulatory limits. For a given transmission or sequence of transmissions with desired settings (e.g., F #), one of the regulatory limits may require that the power of the transmitted acoustic energy not exceed a particular value. This acoustic power limit may result in meeting the other regulatory limits. Each transmit pulse and/or sequence is known to satisfy the regulatory limits or is checked prior to transmission. The different transmit characteristics interact. For example, the imaging frame rate is reduced to allow an increase in transmit power within current FDA (Food And Drug Administration) and IEC (International Electrotechnical Commission) limits of mechanical index, thermal index, and transducer temperature.

Satisfying the regulatory limits is based on empirical data. The limits for the parameters (e.g., MI, TI, and Ispta) are compared to measurements performed in water. Water has very small attenuation. To account for the differences in attenuation between water and tissue, the measurements are derated for tissue attenuation at 0.3 dB/cm/MHz. This deration value is overly conservative to account for signal paths that go through low attenuating mediums, like amniotic fluid, or to account for media that could experience a lot of heating like bone. Most ultrasound imaging actually occurs in media that have a much greater attenuation than 0.3 dB/cm/MHz. For example, liver has attenuation values ranging from 0.5-0.9 dB/cm/MHz. Being overly conservative limits the acoustic power, resulting in less depth of field and/or poorer signal-to-noise ratio.

### SUMMARY

By way of introduction, the preferred embodiments described below include methods, systems, instructions, and computer readable media for adaptive regulation of acoustic output power in medical diagnostic ultrasound imaging. Given transmit settings, the acoustic power and/or another setting may be altered to avoid violating a limit set by regulation. The limit is based, at least in part, on an assumed attenuation. By measuring the attenuation, such as by identifying a type of tissue, the deration factor applied to the in-water measurement may be altered or adapted to current imaging. By accounting for the attenuation in the region to be scanned, a greater output power may be possible while still providing the same level of safety.

In a first aspect, a method for adaptive regulation of acoustic output power in medical diagnostic ultrasound imaging is provided. Attenuation in a scan region is measured. A power limit calculation adaptively adjusts as a function of the attenuation. A power limit is applied to a subsequent acoustic transmission, which has a power determined as a function of an output of the adjusted power limit calculation.

In a second aspect, a medical diagnostic imaging system is provided for adaptive regulation of acoustic output power. A transducer is operable to transmit first acoustic energy, limited by a first power limit and having a first power, along a scan line. A processor is operable to determine a type of tissue along the scan line as a function of acoustic response to the acoustic energy and operable to determine a second power different from the first power as a function of the type of tissue and the first power limit. The transducer is operable to transmit second acoustic energy limited by the first power limit. A display is operable to display an image responsive to the acoustic response to the second acoustic energy.

In a third aspect, a computer readable storage medium has stored therein data representing instructions executable by a programmed processor for adaptive regulation of acoustic output power in medical diagnostic ultrasound imaging. The storage medium includes instructions for limiting acoustic output power based on regulations, detecting an actual characteristic of a region to be imaged, altering the limiting of the acoustic output power in response to the actual characteristic, and imaging as a function of the altered limitation where the altered limitation allows greater acoustic output power than the limiting without the altering.

The present invention is defined by the following claims, and nothing in this section should be taken as a limitation on those claims. Further aspects and advantages of the invention are discussed below in conjunction with the preferred embodiments and may be later claimed independently or in combination.

### BRIEF DESCRIPTION OF THE DRAWINGS

FIG. 1 is a flow chart diagram of one embodiment of a method for adaptive regulation of acoustic output power in medical diagnostic ultrasound imaging; and

FIG. 2 is a block diagram of an embodiment of a system for adaptive regulation of acoustic output power in medical diagnostic ultrasound imaging.

### DETAILED DESCRIPTION OF THE PRESENTLY PREFERRED EMBODIMENTS

An ultrasound system adaptively selects a deration value appropriate for the medium being imaged. Better imaging may be achieved due to the adaptation to the type of tissue being imaged. For example, in harmonic imaging, if the output power limit is due to the calculated MI at a depth of 8 cm when using a transmit frequency of 5.0 MHz, then switching from a deration value of 0.3 to 0.5 dB/cm/MHz increases the output power limit by 8 dB. The net result is an increase of 16 dB or more in the generated harmonic signal. In other examples, Doppler modes and acoustic radiation force imaging (ARFI) modes may benefit from adaptive output power regulation.

Adaptive power limit regulation adapts to the types of tissues present in a transmit signal path. For example, anechoic, speckle, specular, or other types of tissue may be present along a scan line. The type of tissue is determined for adapting the power limits. Some possible approaches for determining the type of tissue include using speckle statistics (e.g. Tissue Equalization (TEQ)), a gradient approach (e.g. Real Time Image Enhancement (RTIE)), a signal energy thresholding approach (e.g. Extend or Boost), tissue stiffness (e.g., very stiff=bone, very soft=fluid), or directly measuring the attenuation. Based on the type of tissue or other indictor of attenuation, a different output power limit or limits are automatically recalculated and applied for subsequent transmit events.

The adaptation may be used in other ways. If the newly calculated power limits cause a new parameter to limit the transducer output power (e.g. switching from an Ispta limit to a MI limit), the system may further adapt the F # or other transmit parameters. Altering other transmit parameters in addition to transmit amplitude may achieve even greater gains in the transmit power. In modes like M-mode, spectral Doppler or ARFI, the signal path extends along a line. The signal path may only be determined in a small region (e.g., the line or focal region). The small region covers at least a portion of the signal path and some surrounding regions. Parallel receive processing may be used to rapidly acquire the signal over a 2D or 3D region about the transmit signal path to ensure a safety buffer of tissue types. The tissue classification results (e.g., the determination of the type of tissue) may be used to determine if Tissue Harmonic Imaging (THI) or fundamental mode imaging may be more effective. For example, scan regions with large anechoic regions might benefit more from THI. As the transmit power adaptively changes, the receive gain may adaptively change in an equal and opposite direction to maintain the same overall image brightness to the user.

In one embodiment, experimentation or another process verifies the detection of tissue type across a broad spectrum of tissue types. The FDA may alter the parameter operation to accept an adaptive approach to acoustic output power limits. Rather than using a mean, median, or other actual measure of attenuation, a conservative approach in the adaptation is maintained. For example, the acoustic power limit calculations are only changed when the path is pure speckle and only changed to use a deration value of 0.5 dB/cm/MHz. Adaptation that is more aggressive may be used. Adaptation, providing more acoustic power than would otherwise be available without adaptation, may produce a nice “pop” in image quality, increasing the signal-to-noise ratio and/or scan depth. The adaptation may be used alone or as part of a system for controlling other settings, such as receive gain, mapping, and dynamic range.

The calculation of the MI value is changed as a function of feedback (e.g., at Tx volts=50, MI used to be 1.9, but now the MI values is 0.9, so the voltage may be increased). Changing the deration may also change the peak location. Just altering the MI limit may not account for changes in the peak location. An image associated with a higher MI (e.g., 3.0) might cause concern for users. In alternative embodiments, the regulatory limit is increased (e.g. raise the MI limit from 1.9 to 3.0).

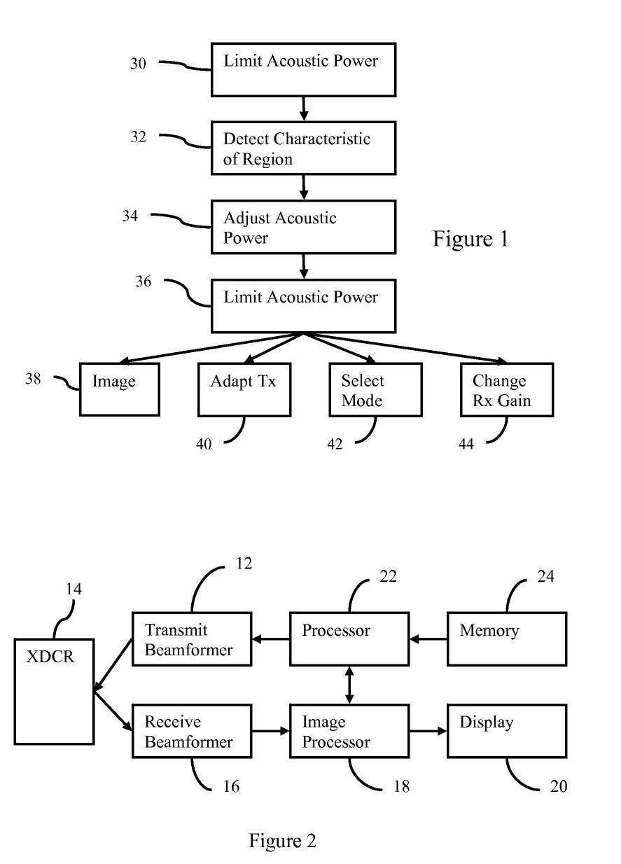

FIG. 1 shows a flowchart of a method for adaptive regulation of acoustic output power in medical diagnostic ultrasound imaging. The method is implemented by the system of FIG. 2, but a different system may be used. Additional, different, or fewer acts may be provided. For example, any one or more of the acts 38-44 related to further use of the adaptation are not performed. As another example, identification of tissue type or other indication of attenuation is provided without the acoustic transmission and associated limiting of act 30. The acts are performed in the order shown or a different order.

In act 30, acoustic output power is limited based on regulations. The transmit sequences have a known or previously measured effect related to a regulatory limit. Alternatively, a program determines the effect given current settings. If the effect will exceed a regulatory limit, such as MI, TI, or Ispta, the acoustic power is reduced. Only transmit sequences that satisfy the regulatory limits are available or may be used.

For an initial or later transmission, adaptation may not be available. The output power limits are set based on an assumed tissue attenuation. Performance of a given transducer and/or imaging system is measured and the assumed tissue attenuation is applied to derate the measurement. The effect, given the current settings, is determined, in part, on the assumed tissue attenuation.

In act 32, an actual characteristic of the region to be imaged is detected. The actual characteristic may not be exact or accurate, such as detecting an approximate characteristic. The actual is an indication of feedback for the current tissue, rather than using an assumed characteristic.

Any characteristic may be detected. For example, a type of tissue is determined. Attenuation may be directly or indirectly determined. For indirect determination, the type of tissue or other measurable characteristic may indicate a likely attenuation. For example, the type of tissue is associated with an attenuation value representing an actual attenuation, such as a conservatively set attenuation specific to the type of tissue as compared to other types of tissue. The characteristic may distinguish between bone, soft tissue, and fluid, and/or between different types of soft tissue.

Possible measurements indicative of the type of tissue and/or attenuation include measuring a speckle characteristic, gradient, signal strength, tissue stiffness, or combination thereof. For example, an intensity signal strength may distinguish between bone, soft tissue, and fluid regions. Bone is highly reflective, so has a higher echo intensity than soft tissue. Soft tissues reflect more acoustic energy than fluids. As another example, the gradient may indicate a speckle change associated with a boundary. Each separate region may be classified after appropriate filtering. Speckle may be smoothed, contrast may be enhanced, or other characteristic emphasized to determine the type of tissue. U.S. Published Patent Application No. 2007/0047788 shows one example. In another example, tissue stiffness, such as measured in strain, shear, or elasticity imaging, indicates the type of tissue. Bone is inelastic as compared to soft tissue. Speckle information may be used to determine a type of tissue in the scan region. For example, soft tissue is distinguished from other types of tissue due to a speckle characteristic, such as shown in U.S. Pat. No. 6,579,238.

The measurement is performed with acoustic energy. The returning echo signals are used to determine the characteristic. Element signals, beamformed signals, and/or detected signals may be used. For example, B-mode intensity information may provide speckle, intensity, or gradient information. Velocity, variance, or motion energy estimates may be used. Acoustic transmissions are used for the measurement. Alternatively, acoustic data acquired for imaging or other purposes is also used to determine the characteristic. In other embodiments, x-ray, magnetic resonance, positron emission, infrared, ultraviolet, or other radiation is used to measure the characteristic.

The characteristic is determined for one or more spatial locations. Any size region may be used. Different types of tissue may be distinguished, such as by border detection. The borders define the regions of different tissue. Alternatively, the type of tissue is determined for each spatial location (e.g., determining the characteristic of the tissue at each ultrasound sample location) or along a tissue type sampling grid (e.g., every 6 samples).

The regions are along one, two or three dimensions. For example, the power limits are to be reset for scanning performed along a single beam, such as associated with M-mode or Spectral Doppler imaging. The characteristic of the tissue along the beam is measured. The characteristic along adjacent beams, such as along one or more adjacent scan lines (receive and/or transmit) on each side of the scan line to be used for imaging, is determined. Measuring the characteristic in adjacent scan lines may account for patient and/or transducer movement, avoiding exceeding power limits for the adjacent region if the beam is repositioned.

As another example, acoustic radiation force imaging, elasticity, shear, color Doppler, or strain imaging may be provided in a more limited region than an entire scan. Acoustic radiation force and/or shear imaging may be performed for a limited number of focal points. Since the acoustic force used to induce motion in tissue likely has a greater amplitude than used for the B-mode or other scanning to measure tissue movement, the variation of the power limits for acoustic force may be handled differently for each type of transmission. The characteristic is determined for the regions subjected to the acoustic force with or without also measuring adjacent regions. The characteristic is determined for less than the entire scan region. In alternative embodiments, the characteristic is determined for the entire scan region, such as an entire B-mode scan region.

In act 34, acoustic power is adjusted. A power limit calculation adaptively adjusts. For adjustment, an old value is altered or a new value is determined or used. Adjusting the calculation may include adjusting a variable used in the calculation, adjusting a look-up table representing the calculation, or selecting a look-up table or formula to be used. For example, a deration value applied by the power limit calculation is adjusted. The deration value is used to select a look-up table or is used as a value in a mathematical formula for calculating a power limit.

The power limit calculation adapts to the measured characteristic, such as tissue type or attenuation. The limiting of the acoustic output power is altered in response to the actual or feedback characteristic. During configuration for scanning and/or real-time scanning, an ultrasound system applies limits. By accounting for the characteristic, the power may be adjusted while satisfying the regulatory limit. The adjustment may allow selection of increased power through a change in amplitude, aperture size, frequency, pulse repetition frequency, and/or other transmit parameter. The regulator limit may be applied merely by allowing or disallowing particular transmit sequences, or by calculating power or other limited values for a given setting.

In one embodiment, an assumed tissue attenuation associated with a power limit (e.g., 0.3 dB/cm/MHz) is altered to another tissue attenuation associated with the type of tissue (e.g., 0.5 dB/cm/MHz). The deration value of 0.3 is changed based on the indication of greater attenuation in the actual scan. The power limit is determined as a function of the actual attenuation. The new deration may reflect a typical attenuation for a given type of tissue or a conservative value that is less than an average, a standard deviation, or minimal attenuation associated with the type of tissue. The new deration is above or below the previous value and/or assumed value.

Satisfaction of the mechanical index, thermal index, intensity spatial peak temporal average another regulated parameter, or combinations thereof are determined as a function of the attenuation or other characteristic of the tissue to be scanned. A value in the determination is altered as a function of the tissue to be subjected to the acoustic transmission. Adaptive adjustment may allow for greater power on transmission.

The adjustment of the power or power limit calculation accounts for the variation of the characteristic determined from the acoustic transmission. For each transmission, the attenuation or other characteristic influencing the power limit is considered. For example, the power at a given location relative to the transducer is different depending on the attenuation of the tissue through which the acoustic energy passes. Wave fronts from different elements pass through the same or different tissue at a same or different distance, so contribute to the total power in different or the same amounts. The power is determined by accounting for all of the tissues through which the acoustic energy passes. Alternatively, the attenuation along the scan line path is used. In other embodiments, a value associated with the least attenuating tissue is used as the attenuation for the entire scan region or acoustic path.

The characteristic of regions outside the scan region or region ideally subjected to an acoustic transmission may be considered in determining the power available under the regulatory power limit. For example, movement may cause the location of transmission to deviate from the intended beam or scan line. Regions along the intended path of acoustic radiation may be included in the calculation. For example, a lowest attenuation along the path and on scan lines adjacent to the intended scan line is used. As another example, the characteristic as a function of space is low pass filtered to determine the characteristic for larger regions. The filtered characteristic is used to determine the power. The power limit calculation adaptively adjusts as a function of attenuation along the scan line and adjacent scan lines.

In act 36, the power is limited, but based on the newly or currently adjusted power limit calculation. After adjustment of the power limit calculation, subsequent transmissions along the scan line, to a region, or over the entire scan region are subject to the corresponding power limit. The power limit calculation and application of the resulting power limit is updated periodically and/or based on a triggering event. Triggering events may include user activation, user changing of imaging parameters, or detection of scanning a different location. Alternatively, the power limit calculation is applied for the remainder of the scanning session and/or current configuration of the ultrasound system.

The power limit is applied in the same or different way than for act 30. A desired transmission or sequence is compared against the power limit and adjusted if the limit would be exceeded. For example, the transmitted power is turned down to meet the regulatory limit. Alternatively, the transmissions and/or sequences available for use are limited to ones that will not exceed the power limit.

One or more, such as all, power limits may be applied. Separate limits on the output power may be calculated for one or more, such as all, of the regulatory parameters. A given transmission or sequence may be limited by one or more of these separate output limits, and not limited by another of them.

Where the power increases due to the adjustment, the acoustic power used for a transmission or sequence of transmissions may be increased or adjusted to be closer to the power limit. The acoustic power of subsequent acoustic transmissions is closer to the power limit, until the power may be updated again. Transmitting with increased power may provide better signal-to-noise ratio, allow for increased scan depth, better move tissue for elasticity, strain, and/or shear wave imaging, or provide another benefit.

In act 38, imaging is performed as a function of the altered acoustic output power. The altered limit calculation allows greater acoustic output power than the limiting without the altering. Acoustic transmissions have greater acoustic output power. The responsive echoes are detected and image processed in any desired manner. B-mode, color Doppler, velocity, variance, energy, M-mode, intensity, contrast agent, harmonic, tissue harmonic, flow, spectral Doppler, three-dimensional rendering, combinations thereof, or other now known or later developed imaging may be used.

In act 40, the acoustic transmissions are further adapted. One or more transmission parameters may be changed to meet one or more regulatory limits. Other transmission parameters may also be changed. For example, the F #, aperture size, pulse length, pulse repetition frequency, frequency, or combinations thereof adapt. Given a new setting allowed by a more inclusive power limit calculation, other parameters of a transmission or sequence may be altered as well. In one embodiment, the adaptation of multiple output power limit calculations results in a different output power limit limiting a subsequent transmission. For example, the newly calculated adapted power causes a new regulatory parameter to limit the transducer output power (e.g. switching from an Ispta-based output power limit to a MI-based output power limit). Other transmission parameters are changed as a function of the change in the type of power limit. The F # or other transmit parameters adapt to achieve even greater gains in the transmit power due to the change in parameters (e.g., since Ispta no longer limits, an increase in pulse repetition rate may be provided without exceeding the MI limit or the adapted Ispta limit).

In act 42, an imaging mode is selected as a function of the characteristic, such as attenuation. The type of tissue may indicate a mode for imaging. For example, a large anechoic region may be imaged with tissue harmonic mode, but soft tissue may be imaged with a fundamental mode. The selection may be a function of other factors, such as one or more power limits.

In act 44, other receive processes adapt. Any receive process may adapt, such as the mode, number of parallel receive beams, filtering, frequency, gain, dynamic range, or combinations thereof. The receive process adapts as a function of the attenuation, the selected imaging mode, adapted power limits, adapted transmission parameters, or combinations thereof. For example, a receive gain adaptively changes as a function of the change to the acoustic power. The overall gain, time gain, or both change in relationship to changes in the transmit power. The amount of change may be for any purpose. In one embodiment, the receive gain changes to maintain an average, median, or other level of brightness. As the transmit power adjusts to a different limit, the receive gain is altered to maintain a similar brightness as the previously generated images. For example, the brightness of a soft tissue region with relatively uniform speckle is maintained at a same level within the dynamic range despite changes in the transmit power. A look-up table, calculation, or offset may be used to determine the gain setting given a change or absolute value of the transmit power.

FIG. 2 shows one embodiment of a medical diagnostic imaging system 10 for adaptive regulation of acoustic output power. The system 10 implements the method of FIG. 1 or other methods. The system 10 includes a transmit beamformer 12, a transducer 14, a receive beamformer 16, an image processor 18, a display 20, a processor 22, and a memory 24. Additional, different or fewer components may be provided. For example, a user input is provided for manual or semi-automated indication of a region of interest and/or triggering adaptation of the power limits. As another example, the processor 22 is part of one of the other components, such as a beamformer controller or the image processor 18.

The transmit beamformer 12 is an ultrasound transmitter, memory, pulser, analog circuit, digital circuit, or combinations thereof. The transmit beamformer 12 is operable to generate waveforms for a plurality of channels with different or relative amplitudes, delays, and/or phasing. Upon transmission of acoustic waves from the transducer 14 in response to the generated waves, one or more beams are formed. A sequence of transmit beams are generated to scan a two or three-dimensional region. Sector, Vector®, linear, or other scan formats may be used. The same region is scanned one time or multiple times. For flow or Doppler imaging and for strain imaging, a sequence of scans to a same region is used. In Doppler imaging and shear velocity estimation, the sequence may include multiple beams along a same scan line before scanning an adjacent scan line. For strain or elasticity imaging, scan or frame interleaving may be used (i.e., scan the entire region before scanning again). In alternative embodiments, the transmit beamformer 12 generates a plane wave or diverging wave for more rapid scanning.

The transmit beams are formed at different energy or amplitude levels. Amplifiers for each channel and/or aperture size control the amplitude of the transmitted beam. Other characteristics may be adjusted, such as the power or frequency of the waveforms.

The transducer 14 is a 1-, 1.25-, 1.5-, 1.75- or 2-dimensional array of piezoelectric or capacitive membrane elements. The transducer 14 includes a plurality of elements for transducing between acoustic and electrical energies. The elements connect with channels of the transmit and receive beamformers 12, 16.

Under the control of the processor 22, the transmit beamformer 12 causes the transducer 14 to transmit acoustic energy. The transmission is along one or more scan lines. The acoustic energy is limited by one or more power limits. The mechanical index, thermal index, Ispta or another regulated parameter controls aspects of the transmitted acoustic energy. The thermal index limits power to avoid generation of possibly destructive heat. The power may be limited to avoid over heating the transducer 14. The limits on the acoustic transmission may change based on the type of tissue being scanned.

Receive signals are generated in response to ultrasound energy (echoes) impinging on the elements of the transducer 14. The receive beamformer 16 includes a plurality of channels with amplifiers, delays, and/or phase rotators, and one or more summers. Each channel connects with one or more transducer elements. The receive beamformer 16 applies relative delays, phases, and/or apodization to form one or more receive beams in response to a transmission. In alternative embodiments, the receive beamformer 16 is a processor for generating samples using Fourier or other transforms.

The receive beamformer 16 may include a filter, such as a filter for isolating information at a second harmonic or other frequency band relative to the transmit frequency band. Such information may more likely include desired tissue, contrast agent, and/or flow information. In another embodiment, the receive beamformer 16 includes a memory or buffer and a filter or adder. Two or more receive beams are combined to isolate information at a desired frequency band, such as a second harmonic, cubic fundamental or other band.

The receive beamformer 16 outputs beam summed data representing spatial locations. Data for a single location, locations along a line, locations for an area, or locations for a volume are output. Dynamic focusing may be provided. The data may be for different purposes. For example, different scans are performed for B-mode or tissue data than for acoustic force radiation imaging. The receive beamformer 16 may include one or more amplifiers for altering a gain of the received signals.

The image processor 18 is a B-mode detector, Doppler detector, pulsed wave Doppler detector, correlation processor, Fourier transform processor, application specific integrated circuit, general processor, control processor, image processor, field programmable gate array, digital signal processor, analog circuit, digital circuit, network, server, group of processors, data path, combinations thereof or other now known or later developed device for detecting and processing information for display from beamformed ultrasound samples. In one embodiment, the processor 18 includes one or more detectors and a separate processor. The image processor 18 detects data from the beamformed samples and generates a medical diagnostic ultrasound image. Filtering, scan conversion, rendering, or other processes may be implemented by the image processor 18.

The processor 22 is an application specific integrated circuit, general processor, control processor, image processor, field programmable gate array, digital signal processor, analog circuit, digital circuit, network, server, group of processors, data path, combinations thereof, or other now known or later developed device for limiting acoustic transmissions. The processor 22 implements the limit calculation to control the transmit beamformer 12. The processor 22 may be a single device or combinations of devices.

The processor 22 receives data from the image processor 18, and/or the receive beamformer 16. The data is detected data, non-detected receive beamformed data, or non-receive beamformed channel data. In other embodiments, the processor 22 receives input from other sources, such as other systems operable to indicate attenuation, such as by classifying tissue type.

The processor 22 determines a type of tissue. The type of tissue is determined from the input data, such as determining as a function of response to transmitted acoustic energy. The type of tissue is determined as a function of a speckle characteristic, gradient, signal strength, tissue stiffness, or combinations thereof. Any indication of the type of tissue or associated attenuation may be used. The type of tissue indicates attenuation. Other measures of attenuation or other characteristics of the region may be determined.

The type of tissue is determined in one or more spatial locations. For example, the type of tissue is determined along one or more scan lines or regions. The type of tissue subjected to a given transmission is determined. The tissue at the focal region, adjacent regions to account for motion, the entire scan region, or other locations may be identified.

The processor 22 determines one or more powers. Different values are determined for the one or more regulated parameters, such as different MI, TI, and/or Ispta values. The processor 22 alters or adapts the power limit calculations to the type of tissue subjected to the acoustic transmission. As the type of tissue changes (e.g., due to motion of the patient or transducer), after initially scanning using an assumed type of tissue (assumed deration value), at specified time intervals, or in response to a trigger event, the processor 22 changes the power or power limit calculation to account for the type of tissue. The power may be greater or lesser over time due to the type of tissue being scanned. For example, the deration value is changed as a function of the type of tissue. The deration value may change from an assumed value to a value selected based on the type of tissue or from a value for one type of tissue or tissue combination to a value for another type of tissue or tissue combination.

The power is determined for each acoustic transmission and/or sequence of transmissions. The power is determined based on the types of tissue along the corresponding scan line or scan lines subjected to the transmission or sequence. The type of tissue from adjacent scan lines or regions may be included in the determination to account for possible transducer movement. For example, a transmit beam does not significantly interact with a fluid tissue region adjacent the scan line for the transmit beam. Since the fluid tissue region is close and may interact with the transmit beam due to unintentional motion, the attenuation associated with the fluid tissue region is considered in determining the amount of attenuation and corresponding power level. The distribution of energy along the beam or relative to each tissue region may also be included in determining the power.

The processor 22 may control other aspects of the transmit beamformer, receive beamformer, and/or image processor. For example, the processor 22 adapts an F #, aperture size, pulse length, pulse repetition frequency, frequency, or combinations thereof as a function of a change in a type of power limit actively limiting a given transmission. As another example, the processor 22 selects between imaging modes as a function of a level or amount of attenuation. In another example, the processor 22 changes a receive gain as a function of a change to acoustic power. The gain counteracts, at least in part, the difference in acoustic energy for different transmissions due to the change in the power limits.

The processor 22 operates pursuant to instructions stored in the memory 24 or another memory. The processor 22 is programmed for adaptive regulation of acoustic output power in medical diagnostic ultrasound imaging. The memory 24 is a computer readable storage media. The instructions for implementing the processes, methods and/or techniques discussed herein are provided on the computer-readable storage media or memories, such as a cache, buffer, RAM, removable media, hard drive or other computer readable storage media. Computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the particular type of instructions set, storage media, processor or processing strategy and may be performed by software, hardware, integrated circuits, firmware, micro code and the like, operating alone or in combination. Likewise, processing strategies may include multiprocessing, multitasking, parallel processing, and the like. In one embodiment, the instructions are stored on a removable media device for reading by local or remote systems. In other embodiments, the instructions are stored in a remote location for transfer through a computer network or over telephone lines. In yet other embodiments, the instructions are stored within a given computer, CPU, GPU or system.

The display device 20 is a CRT, LCD, projector, plasma, printer, or other display for displaying shear velocity, graphics, user interface, validation indication, two-dimensional images, or three-dimensional representations. The display device 20 outputs an image of a region of the patient, such as a two-dimensional elasticity, Doppler tissue, color Doppler, flow, spectral Doppler, M-mode, harmonic, contrast agent, acoustic force radiation, elasticity, strain, shear, or B-mode image. A speaker for outputting audio signals may be provided.

The display device 20 displays ultrasound images and/or other information responsive to transmissions limited by power limits. A sequence of images may be shown, such as in real-time imaging. The images of the sequence are limited by the same or different power limits, based on the same or different regulated parameters. For example, one or more initial images have power limited based on MI, TI, and Ispta, and are determined using an assumed deration value. After determining an amount of attenuation for the scanned region or a subset of the region, the subsequent images may be limited by different values for the power limits or be associated with different power levels due to changing the power calculation for the power limits. Transmissions with different acoustic energy may be used for the different images. The images may include information from different modes of imaging. The transmissions for the different modes may be limited by the same or different power limit values.

The foregoing detailed description has been intended by way of illustration and not limitation. It is only the following claims, including all equivalents that are intended to define the scope of this invention.

1. A method for adaptive regulation of acoustic output power in medical diagnostic ultrasound imaging, the method comprising:
measuring attenuation in a scan region;
adaptively adjusting a power limit calculation as a function of the attenuation; and
applying a power limit to a subsequent acoustic transmission, which has a power determined as a function of an output of the adjusted power limit calculation.

2. The method of claim 1 wherein measuring attenuation in the scan region comprises measuring with acoustic energy.

3. The method of claim 2 wherein measuring comprises measuring a speckle characteristic, gradient, signal strength, tissue stiffness, or combination thereof.

4. The method of claim 1 wherein measuring attenuation comprises determining a type of tissue with the scan region and associating the type of tissue with an attenuation value.

5. The method of claim 1 wherein adaptively adjusting comprises changing a deration value as a function of the attenuation.

6. The method of claim 1 wherein adaptively adjusting comprises determining the power as a function of the attenuation.

7. The method of claim 1 wherein adaptively adjusting comprises adjusting for a mechanical index limit, thermal index limit, intensity spatial peak temporal average limit, or combinations thereof as a function of the attenuation.

8. The method of claim 1 wherein applying comprises adjusting an acoustic power closer to the power limit.

9. The method of claim 1 further comprising adapting an F #, aperture size, pulse length, pulse repetition frequency, frequency, or combinations thereof as a function of a change in a type of power limit.

10. The method of claim 1 wherein measuring comprises measuring along a transmit scan line and adjacent scan lines, wherein adaptively adjusting comprises adaptively adjusting as a function of attenuation along the scan line and adjacent scan lines, and wherein applying comprises applying to the subsequent acoustic transmission along the scan line.

11. The method of claim 1 further comprising selecting between imaging modes as a function of the attenuation.

12. The method of claim 1 wherein applying comprises changing the power of the subsequent acoustic transmission closer to the power limit, and further comprising adaptively changing a receive gain as a function of the change to the acoustic power.

13. A medical diagnostic imaging system for adaptive regulation of acoustic output power, the system comprising:
a transducer operable to transmit first acoustic energy, having a first power limited by a first power limit, along a scan line;
a processor operable to determine a type of tissue along the scan line as a function of acoustic response to the acoustic energy and operable to determine a second power different than the first power as a function of the type of tissue and the first power limit;
wherein the transducer is operable to transmit second acoustic energy limited by the first power limit and at the second power; and
a display operable to display an image responsive to the acoustic response to the second acoustic energy.

14. The medical diagnostic imaging system of claim 13 wherein the processor is operable to determine the type of tissue as a function of a speckle characteristic, gradient, signal strength, tissue stiffness, or combination thereof.

15. The medical diagnostic imaging system of claim 13 wherein the processor is operable to determine the second power by changing a deration value as a function of the type of tissue, the first power limit being based on the mechanical index, thermal index, intensity spatial peak temporal average, or combinations thereof.

16. The medical diagnostic imaging system of claim 13 wherein the processor is operable to adapt an F #, aperture size, pulse length, pulse repetition frequency, frequency, or combinations thereof as a function of a change in a type of power limit.

17. The medical diagnostic imaging system of claim 13 wherein the processor is operable to determine types of tissue along other scan lines adjacent to the scan line, and wherein the processor is operable to determine the second power as a function of the types of tissue along the scan line and other scan lines.

18. The medical diagnostic imaging system of claim 13 wherein the processor is operable to select between imaging modes as a function of the attenuation, and change a receive gain as a function of a change to acoustic power of the second acoustic energy as compared to the first acoustic energy.

19. In a computer readable storage medium having stored therein data representing instructions executable by a programmed processor for adaptive regulation of acoustic output power in medical diagnostic ultrasound imaging, the storage medium comprising instructions for:
limiting acoustic output power based on regulations;
detecting an actual characteristic of a region to be imaged;
altering the limiting of the acoustic output power in response to the actual characteristic; and
imaging as a function of the altered limitation, the altered limitation allowing greater acoustic output power than the limiting without the altering.

20. The computer readable storage medium of claim 19 wherein limiting comprises limiting with an assumed tissue attenuation, wherein detecting comprises detecting a type of tissue, wherein altering the limiting comprises altering the assumed tissue attenuation to another tissue attenuation associated with the type of tissue, and wherein imaging comprises imaging with acoustic transmissions having the greater acoustic output power.
